(19) United States
(12) Patent Application Publication
(10) Pub. No.: US 2023/0307135 A1
(43) Pub. Date: Sep. 28, 2023

(54) AUTOMATED SCREENING FOR DIABETIC RETINOPATHY SEVERITY USING COLOR FUNDUS IMAGE DATA

(71) Applicants: Genentech, Inc., South San Francisco, CA (US); Hoffmann La-Roche Inc., Little Falls, NJ (US)

(72) Inventors: Fethallah BENMANSOUR, Basel (CH); Daniela Ferrara CAVALCANTI, Marlborough, MA (US)

(21) Appl. No.: 18/328,278

(22) Filed: Jun. 2, 2023

Related U.S. Application Data
(63) Continuation of application No. PCT/US2021/061809, filed on Dec. 3, 2021.
(60) Provisional application No. 63/121,711, filed on Dec. 4, 2020.
(60) Provisional application No. 63/169,809, filed on Apr. 1, 2021.

(51) Int. Cl. G16H 50/20; G06T 7/00; A61B 3/12

(52) U.S. Cl. CPC G16H 50/20; G06T 7/0012; A61B 3/12; G06T 2207/20084; G06T 2207/30041

(57) ABSTRACT

Methods and systems for evaluating diabetic retinopathy (DR) severity are provided herein. Color fundus imaging data is received for an eye being evaluated for DR. A metric is generated using the color fundus imaging data, the metric indicating a probability that a score for the DR severity in the eye falls within a selected range.

### CROSS-REFERENCE TO RELATED APPLICATIONS

The present application is a continuation of International Application No. PCT/US2021/061809, filed on Dec. 3, 2021, which claims priority to U.S. Provisional Patent Application No. 63/121,711, filed on Dec. 4, 2020, entitled “AUTOMATED SCREENING FOR DIABETIC RETINOPATHY SEVERITY USING COLOR FUNDUS IMAGE DATA” and to U.S. Provisional Patent Application No. 63/169,809, filed on Apr. 1, 2021, entitled “AUTOMATED SCREENING FOR DIABETIC RETINOPATHY SEVERITY USING COLOR FUNDUS IMAGE DATA,” which applications are incorporated herein by reference in their entireties for all purposes.

### FIELD

This description is generally directed towards evaluating the severity of diabetic retinopathy (DR) in subjects. More specifically, this description provides methods and systems for screening, via a neural network system, for mild to moderate DR, mild to moderately severe DR, mild to severe DR, moderate to moderately severe DR, moderate to severe DR, moderately severe to severe DR, more than mild DR, more than moderate DR, more than moderately severe DR, or more than severe DR using color fundus imaging data.

### BACKGROUND

Diabetic retinopathy (DR) is a common microvascular complication in subjects with diabetes mellitus. DR occurs when high blood sugar levels cause damage to blood vessels in the retina. The two stages of DR include the earlier stage, non-proliferative diabetic retinopathy (NPDR), and the more advanced stage, proliferative diabetic retinopathy (PDR), With NPDR, tiny blood vessels may leak and cause the retina and/or macula to swell. In some cases, macular ischemia may occur, tiny exudates may form in the retina, or both. With PDR, new, fragile blood vessels may grow in a manner that can leak blood into the vitreous humor, damage the optic nerve, or both. Untreated, PDR can lead to severe vision loss and even blindness.

In certain cases, it may be desirable to identify subjects having mild NPDR, moderate NPDR, moderately severe NPDR, or severe NPDR, a level of severity that is on the cusp of advancing to PDR. For example, a clinical trial may use screening exams to identify subjects having moderate NPDR, moderately severe NPDR, or severe NPDR for potential inclusion in the clinical trial. However, some currently available methodologies for performing such screening may require highly trained raters (e.g., human graders, examiners, pathologists, clinicians, rating facilities, rating entities, etc.) and may be inaccurate, rater-dependent, and time-consuming. Thus, there is a need for systems and methods that accurately evaluate DR severity using automated procedures.

### SUMMARY

The present disclosure provides systems and methods for evaluating diabetic retinopathy (DR) severity. Color fundus imaging data is received for an eye being evaluated for DR. A metric is generated using the color fundus imaging data, the metric indicating a probability that a score for the DR severity in the eye falls within a selected range. The output is generated using a trained neural network.

In one or more embodiments, a method is provided for evaluating DR severity. Color fundus imaging data is received for an eye of a subject. A predicted DR severity score for the eye is generated, (for instance, via a neural network system) using the received color fundus imaging data. A metric indicating that the predicted DR severity score falls within a selected range is generated via the neural network system.

### BRIEF DESCRIPTION OF THE DRAWINGS

For a more complete understanding of the principles disclosed herein, and the advantages thereof, reference is now made to the following descriptions taken in conjunction with the accompanying drawings, in which:

FIG. 1 is a block diagram of a first evaluation system, in accordance with various embodiments.

FIG. 2 shows an example of diabetic retinopathy severity scores (DRSS), in accordance with various embodiments.

FIG. 3 shows an example of an image standardization procedure, in accordance with various embodiments

FIG. 4 is a flowchart of a first process for evaluating diabetic retinopathy (DR) severity, in accordance with various embodiments.

FIG. 5 is a block diagram of a second evaluation system, in accordance with various embodiments.

FIG. 6 is a flowchart of a second process for evaluating DR severity, in accordance with various embodiments.

FIG. 7 is a block diagram of a neural network training procedure for use in training the systems described herein with respect to FIGS. 1 and/or 5, in accordance with various embodiments.

FIG. 8 is a block diagram of a computer system, in accordance with various embodiments.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

### Overview

Current methods for screening for subjects who have or are at risk of having diabetic retinopathy (DR) rely on the Diabetic Retinopathy Severity Scale (DRSS) developed by the Early Treatment Diabetic Retinopathy Study (ETDRS). The DRSS is largely considered the gold standard, especially in the research context, for classifying the severity of DR. A DRSS score of 35 indicates mild DR, a DRSS score of 43 indicates moderate DR, a DRSS score of 47 indicates moderately severe DR, and a DRSS score of 53 indicates severe DR, which is the precursor to PDR.

In some cases, a clinical trial or study may be designed for subjects having DR that falls within a selected range of severity. For example, a particular clinical trial may want to focus on subjects having DR that falls between mild and moderate, between mild and moderately severe, between mild and severe, between moderate and moderately severe, between moderately severe and severe between moderate and severe, more than mild, more than moderate, more than moderately severe, or more than severe. Being able to quickly, efficiently, and accurately identify whether a subject's DR can be classified as moderate, moderately severe, or severe may be important to screening or prescreening large numbers of potential subjects.

Currently, screening or prescreening of a subject may include generating one or more color fundus images for a subject and sending those color fundus images to expert human graders who have the requisite knowledge and experience to assign a subject a DRSS score. Repeating this process for hundreds, thousands, or tens of thousands of subjects that may need to undergo screening may be expensive, rater-dependent, and time-consuming. In some cases, this type of manual grading of DR severity in the screening or prescreening process may form a “bottleneck” that may impact the clinical trial or study in an undesired manner. Further, in certain cases, this type of manual grading may not be as accurate as desired due to human error.

Thus, the methods and systems of the present disclosure enable automated screening for selected ranges of diabetic DR. The methods and systems described herein may help reduce the time and costs associated with screening, provide a rater-independent or near rater-independent process, mitigate one or more of the other issues described above, or a combination thereof. In the various embodiments described herein, a neural network system receives color fundus imaging data for an eye of a subject. The neural network system is used to generate an indication of whether a severity score for the DR in the eye falls within a selected range. The selected range may be, for example, but is not limited to, a DRSS score between and including 35 and 43, between and including 35 and 47, between and including 35 and 53, between and including 43 and 47, between and including 47 and 53, between and including 43 and 53, at least 35, at least 43, at least 47, or at least 53.

The neural network is trained using a sufficient number of samples to ensure desired accuracy. In one or more embodiments, the training is performed using samples that have been graded by a single human grader or organization of graders. In other embodiments, the training may be performed using samples that have been graded by multiple human graders or multiple organizations of graders.

Recognizing and taking into account the importance and utility of a methodology and system that can provide the improvements described above, the specification describes various embodiments for evaluating the severity of diabetic retinopathy. More particularly, the specification describes various embodiments of methods and systems for identifying, via a neural network system, whether an eye has mild to moderate DR, mild to moderately severe DR, mild to severe DR, moderate to moderately severe DR, moderate to severe DR, moderately severe to severe DR, more than mild DR, more than moderate DR, more than moderately severe DR, or more than severe DR using color fundus imaging data.

The systems and methods described herein may enable DR that falls within a selected range of severity to be more accurately and quickly identified. This type of rapid identification may improve DR screening, enabling a greater number of subjects to be reliably screened in a shorter amount of time. In some embodiments, improved DR screening may allow healthcare providers to provide improved treatment recommendations or to recommend follow-on risk analysis or monitoring of a subject identified as likely to develop DR. In some embodiments, the systems and methods described herein may be used to train expert human graders to more accurately and efficiently identify DR that falls within a selected range of severity or to flag eyes of subjects that may have DR for further analysis by expert human graders. In some embodiments, the systems and methods described herein may be used to accurately and efficiently select subjects for inclusion in clinical trials. For instance, if a clinical trial aims to treat subjects who have or are at risk of having a particular DR severity (such as mild to moderate DR, mild to moderately severe DR, mild to severe DR, or any other DR severity described herein), the systems and methods can be used to identify only those subject who have or are at risk of developing that DR severity for inclusion in the clinical trial.

### Definitions

The disclosure is not limited to these exemplary embodiments and applications or to the manner in which the exemplary embodiments and applications operate or are described herein. Moreover, the figures may show simplified or partial views, and the dimensions of elements in the figures may be exaggerated or otherwise not in proportion.

In addition, as the terms “on,” “attached to,” “connected to,” “coupled to,” or similar words are used herein, one element (e.g., a component, a material, a layer, a substrate, etc.) can be “on,” “attached to,” “connected to,” or “coupled to” another element regardless of whether the one element is directly on, attached to, connected to, or coupled to the other element or there are one or more intervening elements between the one element and the other element. In addition, where reference is made to a list of elements (e.g., elements a, b, c), such reference is intended to include any one of the listed elements by itself, any combination of less than all of the listed elements, and/or a combination of all of the listed elements. Section divisions in the specification are for ease of review only and do not limit any combination of elements discussed.

The term “subject” may refer to a subject of a clinical trial, a person undergoing treatment, a person undergoing anti-cancer therapies, a person being monitored for remission or recovery, a person undergoing a preventative health analysis (e.g., due to their medical history), or any other person or patient of interest. In various cases, “subject” and “patient” may be used interchangeably herein.

Unless otherwise defined, scientific and technical terms used in connection with the present teachings described herein shall have the meanings that are commonly understood by those of ordinary skill in the art. Further, unless otherwise required by context, singular terms shall include pluralities and plural terms shall include the singular. Generally, nomenclatures utilized in connection with, and techniques of, chemistry, biochemistry, molecular biology, pharmacology and toxicology are described herein are those well-known and commonly used in the art.

As used herein, “substantially” means sufficient to work for the intended purpose. The term “substantially” thus allows for minor, insignificant variations from an absolute or perfect state, dimension, measurement, result, or the like such as would be expected by a person of ordinary skill in the field but that do not appreciably affect overall performance. When used with respect to numerical values or parameters or characteristics that can be expressed as numerical values, “substantially” means within ten percent.

The term “ones” means more than one.

As used herein, the term “plurality” can be 2, 3, 4, 5, 6, 7, 8, 9, 10, or more.

As used herein, the term “set of” means one or more. For example, a set of items includes one or more items.

As used herein, the phrase “at least one of,” when used with a list of items, means different combinations of one or more of the listed items may be used and only one of the items in the list may be needed. The item may be a particular object, thing, step, operation, process, or category. In other words, “at least one of” means any combination of items or number of items may be used from the list, but not all of the items in the list may be required. For example, without limitation, “at least one of item A, item B, or item C” means item A; item A and item B; item B; item A, item B, and item C; item B and item C; or item A and C. In some cases, “at least one of item A, item B, or item C” means, but is not limited to, two of item A, one of item B, and ten of item C; four of item B and seven of item C; or some other suitable combination.

As used herein, a “model” may include one or more algorithms, one or more mathematical techniques, one or more machine learning algorithms, or a combination thereof.

As used herein, “machine learning” includes the practice of using algorithms to parse data, learn from it, and then make a determination or prediction about something in the world. Machine learning uses algorithms that can learn from data without relying on rules-based programming.

As used herein, an “artificial neural network” or “neural network” (NN) may refer to mathematical algorithms or computational models that mimic an interconnected group of artificial neurons that processes information based on a connectionistic approach to computation. Neural networks, which may also be referred to as neural nets, can employ one or more layers of nonlinear units to predict an output for a received input. Some neural networks include one or more hidden layers in addition to an output layer. The output of each hidden layer is used as input to the next layer in the network, i.e., the next hidden layer or the output layer. Each layer of the network may generate an output from a received input in accordance with current values of a respective set of parameters. In the various embodiments, a reference to a “neural network” may be a reference to one or more neural networks.

A neural network may process information in two ways; when it is being trained it is in training mode and when it puts what it has learned into practice it is in inference (or prediction) mode. Neural networks learn through a feedback process (e.g., backpropagation) which allows the network to adjust the weight factors (modifying its behavior) of the individual nodes in the intermediate hidden layers so that the output matches the outputs of the training data. In other words, a neural network learns by being fed training data (learning examples) and eventually learns how to reach the correct output, even when it is presented with a new range or set of inputs. A neural network may include, for example, without limitation, at least one of a Feedforward Neural Network (FNN), a Recurrent Neural Network (RNN), a Modular Neural Network (MNN), a Convolutional Neural Network (CNN), a Residual Neural Network (ResNet), an Ordinary Differential Equations Neural Networks (neural-ODE), or another type of neural network.

### Automated Screening for Diabetic Retinopathy Severity

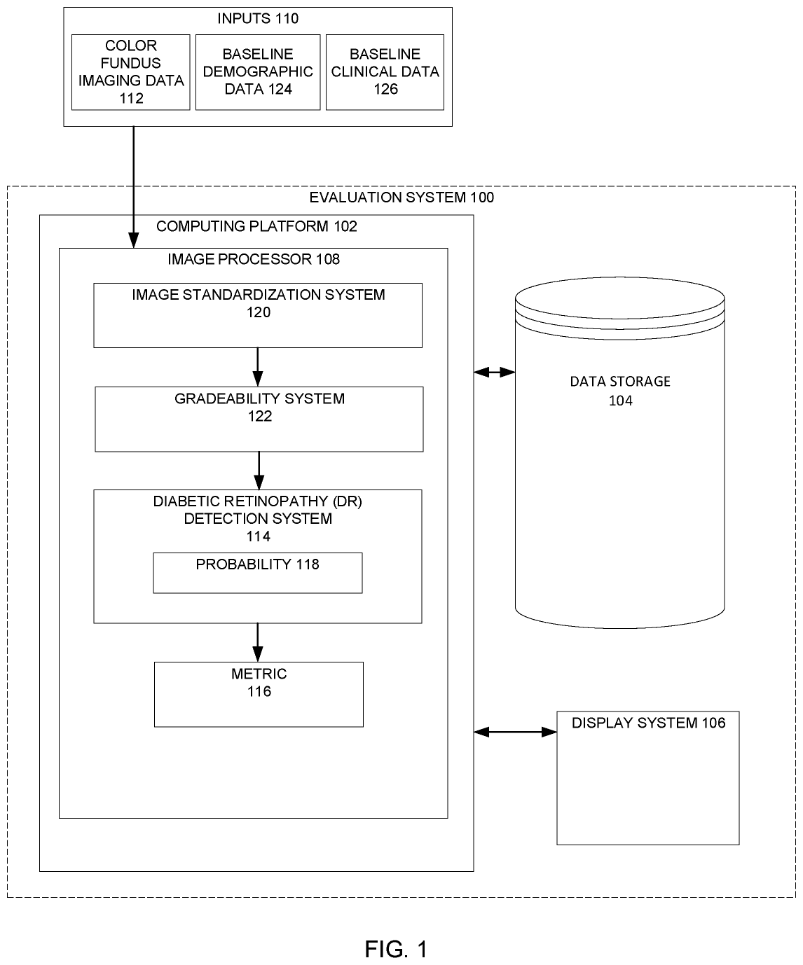

FIG. 1 is a block diagram of a first evaluation system 100 in accordance with various embodiments. Evaluation system 100 is used to evaluate diabetic retinopathy (DR) severity in one or more eyes (for instance, one or more retinas) of one or more subjects.

Evaluation system 100 includes computing platform 102, data storage 104, and display system 106. Computing platform 102 may take various forms. In one or more embodiments, computing platform 102 includes a single computer (or computer system) or multiple computers in communication with each other. In other examples, computing platform 102 takes the form of a cloud computing platform.

Data storage 104 and display system 106 are each in communication with computing platform 102. In some examples, data storage 104, display system 106, or both may be considered part of or otherwise integrated with computing platform 102. Thus, in some examples, computing platform 102, data storage 104, and display system 106 may be separate components in communication with each other, but in other examples, some combination of these components may be integrated together.

Evaluation system 100 includes image processor 108, which may be implemented using hardware, software, firmware, or a combination thereof. In one or more embodiments, image processor 108 is implemented in computing platform 102.

Image processor 108 receives input 110 for processing. In one or more embodiments, input 110 includes color fundus imaging data 112. Color fundus imaging data 112 may include, for example, one or a plurality of fields of view (or fields) of color fundus images generated using a color fundus imaging technique (also referred to as color fundus photography). In one or more embodiments, color fundus imaging data 112 includes seven-field color fundus imaging data. In some embodiments, each field of view comprises a color fundus image.

Image processor 108 processes at least color fundus imaging data 112 of input 110 using DR detection system 114 to generate a metric 116. In some embodiments, DR detection system 114 comprises a neural network system. In one or more embodiments, the metric 116 indicates a probability 118 that a score for the DR severity (e.g., a DRSS score) in the eye falls within a selected range. The selected range may be, for example, but is not limited to, a mild to moderate range, a mild to moderately severe range, a mild to severe range, a moderate to moderately severe range, a moderately severe to severe range, a moderate to severe range, a more than mild range, a more than moderate range, a more than moderately severe range, or a more than severe range. In one or more embodiments, these ranges correspond to a portion of the DRSS between and including 35 and 43, between and including 35 and 47, between and including 35 and 53, between and including 43 and 47, between and including 47 and 53, between and including 43 and 53, at least 35, at least 43, at least 47, or at least 53, respectively.

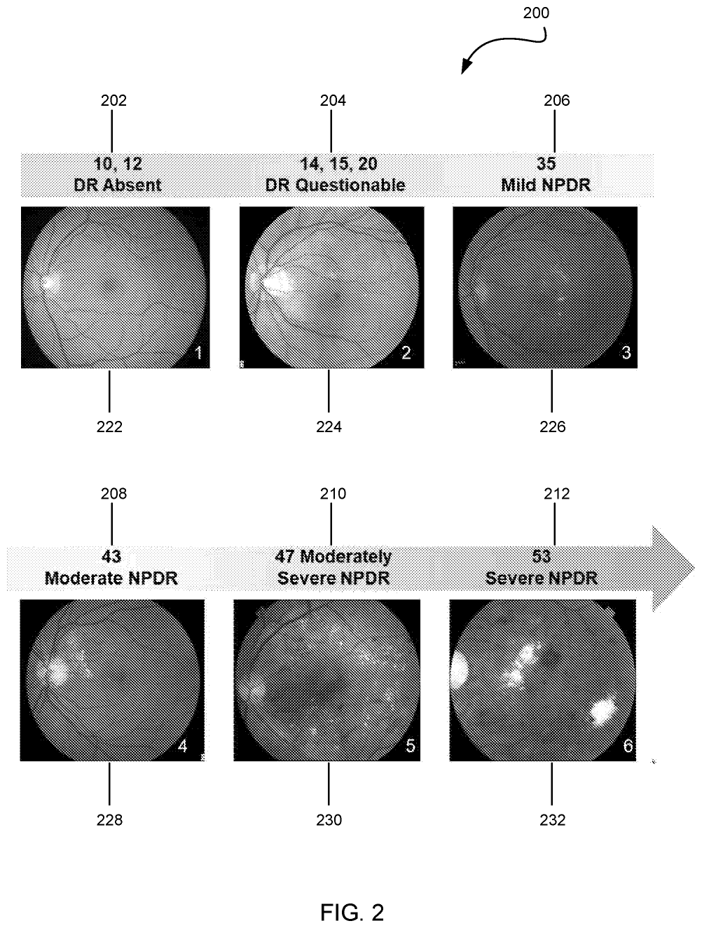

FIG. 2 shows an example 200 of DRSS scores in accordance with various embodiments. In some embodiments, a first score 202 between and including 10 and 12 indicates that DR is absent from an eye. In some embodiments, a second score 204 between and including 14 and 20 indicates that DR may be present in the eye (i.e., DR is questionable in the eye). In some embodiments, a third score 206 of at least 35 or of between and including 35 and 43 indicates that mild DR may be present in the eye. In some embodiments, a fourth score 208 of at least 43 or of between and including 43 and 47 indicates that moderate DR may be present in the eye. In some embodiments, a fifth score 210 of at least 47 or of between and including 47 and 53 indicates that moderately severe DR may be present in the eye. In some embodiments, a sixth score 212 of at least 53 indicates that moderately sever DR may be present in the eye. FIG. 2 further shows an exemplary first fundus image 222 associated with the first score, an exemplary second fundus image 224 associated with the second score, an exemplary third fundus image 226 associated with the third score, an exemplary fourth fundus image 228 associated with the fourth score, an exemplary fifth fundus image 230 associated with the fifth score, and an exemplary sixth image 232 associated with the sixth score.

Returning to the discussion of FIG. 1, in some embodiments, metric 116 takes the form of a probability value between and/or including 0 and 1. In other embodiments, metric 116 is a category or classifier for the probability (e.g., a category selected from a low probability and a high probability, etc.). In one or more embodiments, metric 116 is a binary indication of whether the probability is above a selected threshold. In some embodiments, the threshold is at least about 0.5, 0.6, 0.7, 0.8, 0.9, or more. In some embodiments, the threshold is at most about 0.9, 0.8, 0.7, 0.6, 0.5, or less. In some embodiments, the threshold is within a range defined by any two of the preceding values.

In other embodiments, image processor 108 processes at least color fundus imaging data 112 of input 110 using DR detection system 114 to generate a predicted DR severity score (e.g., a predicted DRSS score). Image processor 108 may then generate metric 116 indicating the probability 118 that the predicted diabetic retinopathy severity score falls within the selected range.

DR detection system 114 may include any number of or combination of neural networks. In one or more embodiments, DR detection system 114 takes the form of a convolutional neural network (CNN) system that includes one or more neural networks. Each of these one or more neural networks may itself be a convolutional neural network.

In some embodiments, image processor 108 further comprises an image standardization system 120. In some embodiments, the image standardization system 120 is configured to perform at least one image standardization procedure on the color fundus imaging data 112 to generate a set of standardized image data. In some embodiments, the at least one image standardization procedure comprises one or more of: a field detection procedure, a central cropping procedure, a foreground extraction procedure, a region extraction procedure, a central region extraction procedure, an adaptive histogram equalization (AHE) procedure, and a contrast limited AHE (CLAHE) procedure. In some embodiments, the image standardization system 120 is configured to perform any at least 1, 2, 3, 4, 5, 6, or 7, or at most any 7, 6, 5, 4, 3, 2, or 1 of the aforementioned procedures.

In some embodiments, a field detection procedure comprises any procedure configured to detect a field of view within a color fundus image from which features of the color fundus image are to be extracted. In some embodiments, a central cropping procedure comprises any procedure configured to crop a central region of the color fundus image from the remainder of the color fundus image. In some embodiments, a foreground extraction procedure comprises any procedure configured to extract a foreground region of the color fundus image from the remainder of the color fundus image. In some embodiments, a region extraction procedure comprises any procedure configured to extract any region of the color fundus image from the remainder of the color fundus image. In some embodiments, a central region extraction procedure comprises any procedure configured to extract a central region of the color fundus image from the remainder of the color fundus image.

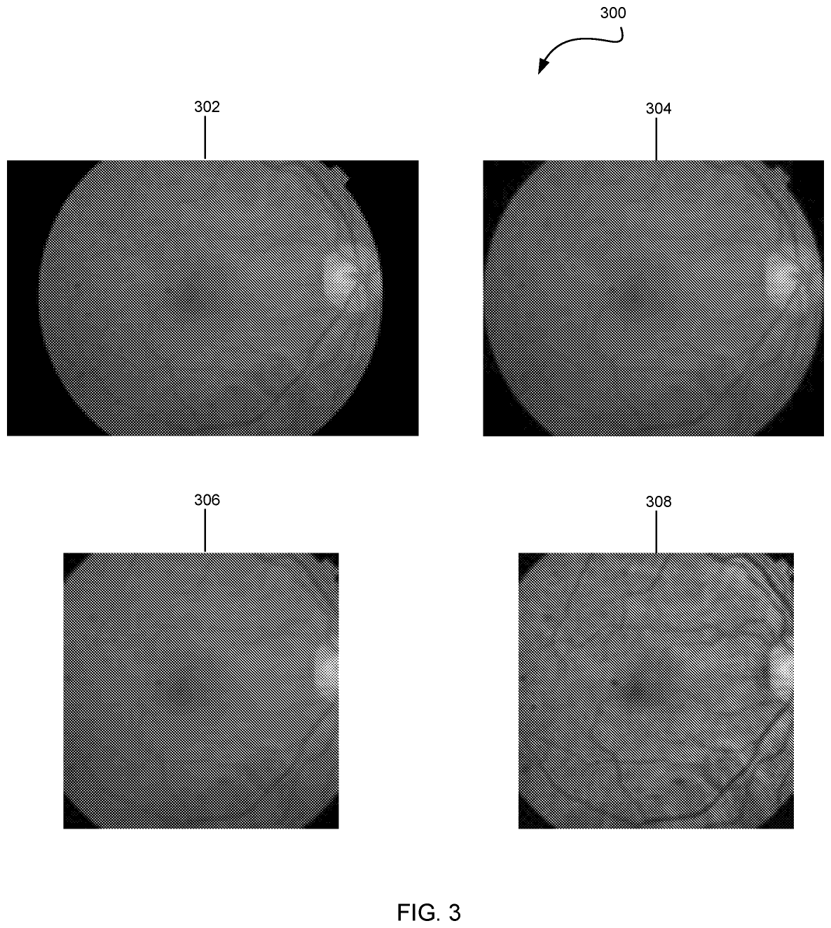

FIG. 3 shows an example of an image standardization procedure 300. In the example shown, an input color fundus image 302 of an eye is received.

Next, a foreground region of the eye (such as a fundus subregion of the eye) is extracted to generate a foreground image 304 of the eye from the input color fundus image 302. In some embodiments, the foreground region is extracted using a field detection procedure or a foreground extraction procedure. In some embodiments, the foreground region is extracted by constructing a binary mask in the color fundus image. In some embodiments, the binary mask is retrieved from the input color fundus image using an intensity thresholding operation. In some embodiments, the threshold is estimated from at least one, two, three, or four corners of the input color fundus image or at most four, three, two, or one corners of the input color fundus image. In some embodiments, when the input color fundus image contains text or labels in one or more corners, at least one or two, or most one or two of the brightest corners in the image are excluded and the remaining corners are used to estimate the threshold. In some embodiments, the threshold is increased by a factor in order to ensure that substantially all pixels in the foreground region of the eye are included in the foreground image 304. In some embodiments, the factor is determined experimentally. In some embodiments, the binary mask is then replaced by the largest connected component of the binary mask that does not include a background region of the input color fundus image. In some embodiments, a binary dilation is performed on the binary mask in order to fill holes in the binary mask.

A central region of the eye may be extracted to generate a central region image 306 of the eye from the foreground image 304. In some embodiments, the central region is extracted using a central cropping procedure, a region extraction procedure, or a central region extraction procedure. In some embodiments, the central region is extracted using a Hough transform or circular Hough transform.

A contrast enhancement procedure may also be applied to generate a contrast-enhanced image 308 of the eye from the central region image 306. In some embodiments, the contrast enhancement procedure comprises an AHE procedure or a CLAHE procedure.

In some embodiments, the image standardization procedure 300 produces standardized image data that improves performance of the system 100 (described herein with respect to FIG. 1) when compared to use of the system 100 on raw color fundus imaging data. In some embodiments, the standardized image data is used to generate the metric 116 (described herein with respect to FIG. 1).

Returning to the discussion of FIG. 1, in some embodiments, image processor 108 further comprises a gradeability system 122. In some embodiments, gradeability system 122 is configured to determine a gradeability of the color fundus imaging data (or the standardized image data) based on a number of fields of view associated with the color fundus imaging data. In some embodiments, the color fundus imaging data (or the standardized image data) may contain a number of fields of view that is insufficient to determine a DRSS. For instance, color fundus imaging data that is used to detect clinically significant macular edema (CSME) may only contain one field of view and may therefore not contain information sufficient to determine a DRSS. Thus, the gradeability may indicate that the color fundus imaging data (or the standardized image data) does or does not contain at least a predetermined number of fields of view. In some embodiments, the predetermined number is at least about 2, 3, 4, 5, 6, 7, 8, or more, at most 8, 7, 6, 5, 4, 3, or 2, or within a range defined by any two of the preceding values. In some embodiments, the gradeability system 122 is configured to filter out color fundus imaging data (or standardized image data) that does not contain at least the predetermined number of fields of view.

In some embodiments, the gradeability system 122 is configured to receive input from the image standardization system 120, as shown in FIG. 1. In some embodiments, the image standardization system 120 is configured to receive input from the gradeability system 122.

In some embodiments, the input data 110 further comprises baseline demographic data 124 associated with the subject and/or baseline clinical data 126 associated with the subject. In some embodiments, the baseline demographic data 124 comprises an age, sex, height, weight, race, ethnicity, and/or other demographic data associated with the subject. In some embodiments, the baseline clinical data 126 comprises a diabetic status of the subject, such as a diabetes type (e.g., type 1 diabetes or type 2 diabetes) or diabetes duration. In some embodiments, the metric 116 is generated using the baseline demographic data and/or the baseline clinical data in addition to the color fundus imaging data.

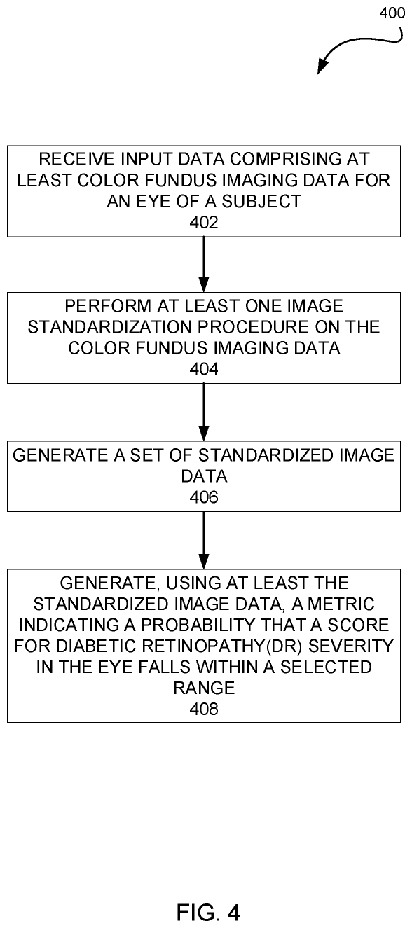

FIG. 4 is a flowchart of a first process 400 for evaluating DR severity in accordance with various embodiments. In one or more embodiments, process 400 is implemented using the evaluation system 100 described in FIG. 1.

Step 402 includes receiving input data comprising at least color fundus imaging data for an eye of a subject. The color fundus imaging data for the eye may comprise any color fundus imaging data described herein with respect to FIG. 1.

Step 404 includes performing at least one image standardization procedure on the color fundus imaging data. The at least one image standardization procedure may be any image standardization procedure described herein with respect to FIG. 1 or 3.

Step 406 includes generating a set of standardized image data. In some embodiments, the standardized image data is generated using the at least one image standardization procedure.

Step 408 includes generating, using at least the standardized image data, a metric indicating a probability that a score for the DR severity in the eye falls within a selected range. This metric may be, for example, metric 116 described herein with respect to FIG. 1. The selected range may be any selected range described herein with respect to FIG. 1. In some embodiments, the metric is generated using a neural network system, such as DR detection system 114 described herein with respect to FIG. 1.

In some embodiments, the method further comprises determining a gradeability of the color fundus imaging data, as described herein with respect to FIG. 1. In some embodiments, the method further comprises filtering out the color fundus imaging data if it does not contain at least a predetermined number of fields of view, as described herein with respect to FIG. 1.

In some embodiments, the input data further comprises any baseline demographic data and/or baseline clinical data associated with the subject, as described herein with respect to FIG. 1. In some embodiments, the metric is generated using the baseline demographic data and/or baseline clinical data in addition to the color fundus imaging data, as described herein with respect to FIG. 1.

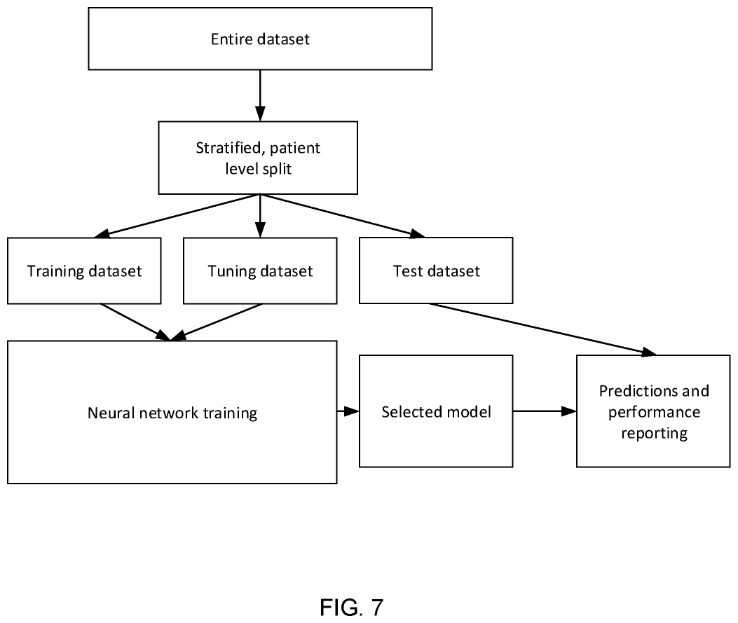

In some embodiments, the method further comprises training a neural network system using a training dataset comprising at least graded color fundus imaging data associated with a plurality of training subjects. In some embodiments, the training dataset further comprises baseline demographic data associated with the plurality of training subjects and/or baseline clinical data associated with the plurality of training subjects. The plurality of training subjects may comprise any number of subjects, such as at least about 1 thousand, 2 thousand, 3 thousand, 4 thousand, 5 thousand, 6 thousand, 7 thousand, 8 thousand, 9 thousand, 10 thousand, 20 thousand, 30 thousand, 40 thousand, 50 thousand, 60 thousand, 70 thousand, 80 thousand, 90 thousand, 100 thousand, 200 thousand, 300 thousand, 400 thousand, 500 thousand, 600 thousand, 700 thousand, 800 thousand, 900 thousand, 1 million, or more subjects, at most about 1 million, 900 thousand, 800 thousand, 700 thousand, 600 thousand, 500 thousand, 400 thousand, 300 thousand, 200 thousand, 100 thousand, 90 thousand, 80 thousand, 70 thousand, 60 thousand, 50 thousand, 40 thousand, 30 thousand, 20 thousand, 10 thousand, 9 thousand, 8 thousand, 7 thousand, 6 thousand, 5 thousand, 4 thousand, 3 thousand, 2 thousand, 1 thousand, or fewer subjects, or a number of subjects that is within a range defined by any two of the preceding values. In some embodiments, the method further comprises training the neural network system using the method described herein with respect to FIG. 7.

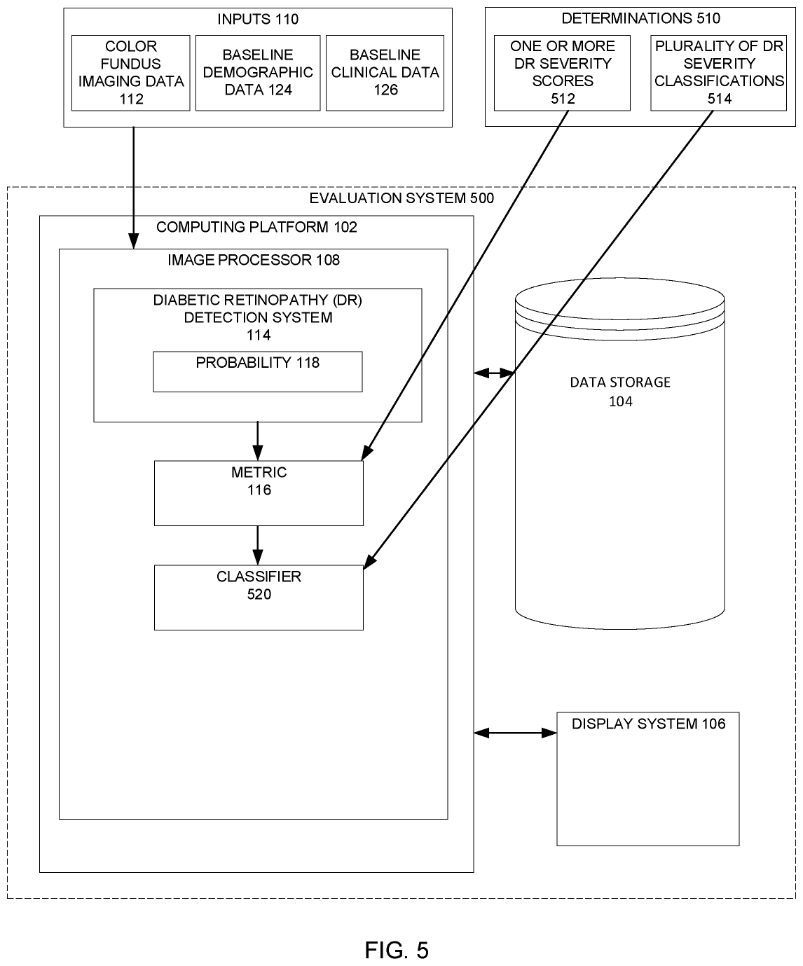

FIG. 5 is a block diagram of a second evaluation system 500 in accordance with various embodiments. Evaluation system 500 is used to evaluate diabetic retinopathy (DR) severity in one or more eyes (for instance, one or more retinas) of one or more subjects.

Evaluation system 500 may be similar to evaluation system 100 described herein with respect to FIG. 1. In some embodiments, evaluation system 500 includes computing platform 102, data storage 104, display system 106, and image processor 108, as described herein with respect to FIG. 1. In some embodiments, image processor 108 receives input 110 for processing, as described herein with respect to FIG. 1. In some embodiments, input 110 includes color fundus imaging data 112, as described herein with respect to FIG. 1. Image processor 108 processes at least color fundus imaging data 112 of input 110 using DR detection system 114 to generate metric 116, as described herein with respect to FIG. 1. In one or more embodiments, the metric 116 indicates a probability 118 that a score for the DR severity (e.g., a DRSS score) in the eye falls within a selected range, as described herein with respect to FIG. 1. The selected range may be any selected range described herein with respect to FIG. 1. In some embodiments, input 100 includes baseline demographic data 124 and/or baseline clinical data 126, as described herein with respect to FIG. 1.

In comparison with the first evaluation system 100, evaluation system 500 may be configured to receive one or more determinations 510 that may be used to determine the metric and/or a classification of the eye. In some embodiments, the one or more determinations are provided by an expert or associated with an expert determination. In some embodiments, the one or more determinations include one or more DR severity scores 512. In some embodiments, the one or more DR severity scores 512 are based on an expert determination of a DR severity score associated with an eye of a subject. For example, in some embodiments, the one or more DR severity scores are based on an expert grade of a DR severity score associated with the eye of the subject. In some embodiments, the one or more determinations include a plurality of DR severity classifications 514. In some embodiments, the plurality of DR severity classifications 514 are based on an expert determination of DR severity classifications associated with a particular DR severity score. In some embodiments, the plurality of DR severity classifications denote one or more of a mild to moderate DR (corresponding to a DRSS between and including 35 and 43), a mild to moderately severe DR (corresponding to a DRSS between and including 35 and 47), a mild to severe DR (corresponding to a DRSS between and including 35 and 53), a moderate to moderately severe DR (corresponding to a DRSS between and including 43 and 47), a moderate to severe DR (corresponding to a DRSS between and including 43 and 53), a moderately severe to severe DR (corresponding to a DRSS between and including 47 and 53), a more than mild DR (corresponding to a DRSS of at least 35), a more than moderate DR (corresponding to a DRSS of at least 43), a more than moderately severe DR (corresponding to a DRSS of at least 47), and a more than severe DR (corresponding to a DRSS of at least 53).

Thus, in some embodiments, the evaluation system 500 is configured to receive a determination of the one or more DR severity scores and to determine the metric based at least in part on this determination.

In some embodiments, the evaluation system 500 further comprises a classifier 520. In some embodiments, the evaluation system is configured to receive a determination of the plurality of DR severity classifications and the classifier is configured to classify the eye into a DR severity classification of the plurality of DR severity classifications based on the metric and the plurality of DR severity classifications.

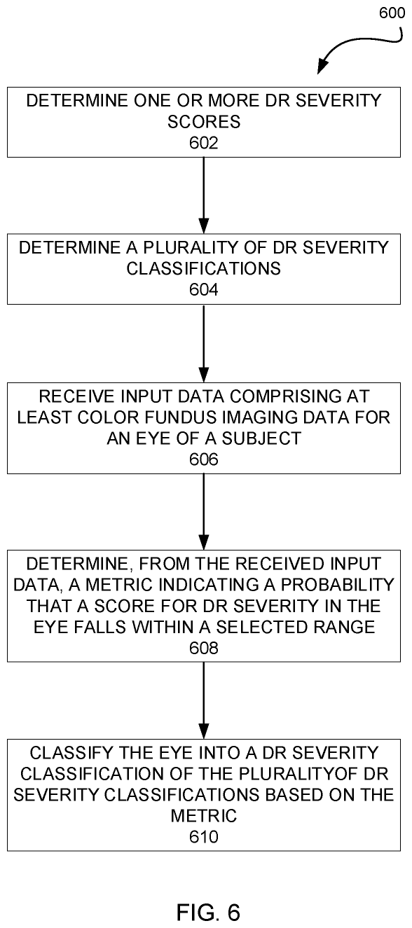

FIG. 6 is a flowchart of a second process 600 for evaluating DR severity in accordance with various embodiments. In one or more embodiments, process 600 is implemented using the evaluation system 500 described in FIG. 5.

Step 602 includes determining one or more DR severity scores. In some embodiments, each score is associated with a DR severity level. The one or more DR severity scores may be any DR severity scores described herein with respect to FIG. 5.

Step 604 includes determining a plurality of DR severity classifications, each classification denoted by a range or a set of DR severity threshold scores. The plurality of DR severity classifications may be any DR severity classifications described herein with respect to FIG. 5.

Step 606 includes receiving input data comprising at least color fundus imaging data for an eye of a subject.

Step 608 includes determining, from the received input data, a metric indicating a probability that a score for DR severity in the eye of the subject falls within a selected range. The selected range may be any selected range described herein with respect to FIG. 5.

Step 610 includes classifying the eye into a DR severity classification of the plurality of DR severity classifications based on the metric.

In some embodiments, the input data further comprises any baseline demographic data and/or baseline clinical data associated with the subject, as described herein with respect to FIG. 1. In some embodiments, the metric is generated using the baseline demographic data and/or baseline clinical data in addition to the color fundus imaging data, as described herein with respect to FIG. 1.

In some embodiments, the method further comprises training a neural network system using a training dataset comprising at least graded color fundus imaging data associated with a plurality of training subjects. In some embodiments, the training dataset further comprises baseline demographic data associated with the plurality of training subjects and/or baseline clinical data associated with the plurality of training subjects. The plurality of training subjects may comprise any number of subjects, such as at least about 1 thousand, 2 thousand, 3 thousand, 4 thousand, 5 thousand, 6 thousand, 7 thousand, 8 thousand, 9 thousand, 10 thousand, 20 thousand, 30 thousand, 40 thousand, 50 thousand, 60 thousand, 70 thousand, 80 thousand, 90 thousand, 100 thousand, 200 thousand, 300 thousand, 400 thousand, 500 thousand, 600 thousand, 700 thousand, 800 thousand, 900 thousand, 1 million, or more subjects, at most about 1 million, 900 thousand, 800 thousand, 700 thousand, 600 thousand, 500 thousand, 400 thousand, 300 thousand, 200 thousand, 100 thousand, 90 thousand, 80 thousand, 70 thousand, 60 thousand, 50 thousand, 40 thousand, 30 thousand, 20 thousand, 10 thousand, 9 thousand, 8 thousand, 7 thousand, 6 thousand, 5 thousand, 4 thousand, 3 thousand, 2 thousand, 1 thousand, or fewer subjects, or a number of subjects that is within a range defined by any two of the preceding values. In some embodiments, the method further comprises training the neural network system using the method described herein with respect to FIG. 7.

FIG. 7 is a block diagram of a neural network training procedure for use in training the DR prediction systems or neural network systems described herein with respect to FIGS. 1 and/or 5. During a learning phase, the neural network system can be trained to determine the metrics and/or classifications described herein with respect to FIGS. 1, 4, 5, and/or 6. In During the training phase, a dataset (such as the graded color fundus imaging data associated with a plurality of training subjects described herein) used to train the neural network system (referred to as an “entire dataset” in FIG. 7) may be first stratified and split at the patient level. The entire dataset may then be divided into a first portion used for training the neural network system (referred to as a “training dataset” in FIG. 7), a second portion used for tuning the neural network (referred to as a “tuning dataset” in FIG. 7), and a third portion that is held out for later testing and/or assessment of the trained neural network system (reference to as a “test dataset” in FIG. 7). The first portion may comprise at least about 70%, 75%, 80%, 85%, 90%, 95%, or more of the entire dataset, at most about 95%, 90%, 85%, 80%, 75%, 70%, or less of the entire dataset, or a percentage of the entire dataset that is within a range defined by any two of the preceding values. The second portion may comprise at least about 5%, 10%, 15%, 20%, or more of the entire dataset, at most about 20%, 15%, 10%, 5%, or less of the entire dataset, or a percentage of the entire dataset that is within a range defined by any two of the preceding values. The third portion may comprise at least about 5%, 10%, 15%, 20%, or more of the entire dataset, at most about 20%, 15%, 10%, 5%, or less of the entire dataset, or a percentage of the entire dataset that is within a range defined by any two of the preceding values.

The training dataset may be used to train the neural network system. The tuning dataset may be used to test and tune the performance of the neural network system following training with the training dataset. The resulting trained neural network system may be applied to the test dataset to predict any metric and/or any classification described herein associated with the test dataset. The predicted metrics and/or classifications may be compared with “ground truths” (such as the actual time-series responses and/or corresponding image features) associated with the holdout dataset using a variety of statistical measures. For instance, the measures may comprise any one or more of an le value, a root-mean squared error (RMSE), a mean absolute error (MAE), and a Pearson correlation coefficient.

### Computer Implemented Systems

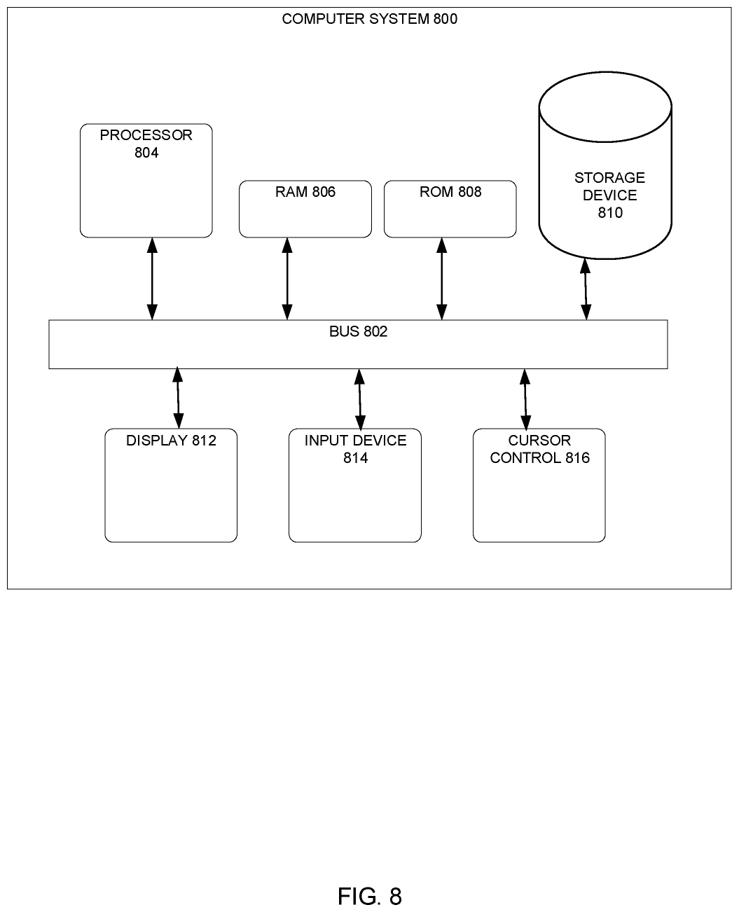

FIG. 8 is a block diagram of a computer system in accordance with various embodiments. Computer system 800 may be an example of one implementation for computing platform 102 described above in FIG. 1 and/or FIG. 5. In one or more examples, computer system 800 can include a bus 802 or other communication mechanism for communicating information and at least one processor 804 coupled with bus 802 for processing information. In various embodiments, computer system 800 can also include a memory, which can be a random-access memory (RAM) 806 or other dynamic storage device, coupled to bus 802 for determining instructions to be executed by processor 804. Memory also can be used for storing temporary variables or other intermediate information during execution of instructions to be executed by processor 804. In various embodiments, computer system 800 can further include a read only memory (ROM) 808 or other static storage device coupled to bus 802 for storing static information and instructions for processor 804. A storage device 810, such as a magnetic disk or optical disk, can be provided and coupled to bus 802 for storing information and instructions.

In various embodiments, computer system 800 can be coupled via bus 802 to a display 812, such as a cathode ray tube (CRT) or liquid crystal display (LCD), for displaying information to a computer user. An input device 814, including alphanumeric and other keys, can be coupled to bus 802 for communicating information and command selections to processor 804. Another type of user input device is a cursor control 816, such as a mouse, a joystick, a trackball, a gesture input device, a gaze-based input device, or cursor direction keys for communicating direction information and command selections to processor 804 and for controlling cursor movement on display 812. This input device 814 typically has two degrees of freedom in two axes, a first axis (e.g., x) and a second axis (e.g., y), that allows the device to specify positions in a plane. However, it should be understood that input devices 814 allowing for three-dimensional (e.g., x, y and z) cursor movement are also contemplated herein.

Consistent with certain implementations of the present teachings, results can be provided by computer system 800 in response to processor 804 executing one or more sequences of one or more instructions contained in RAM 806. In some embodiments, computer system 800 may provide results in response to one or more special-purpose processing units executing one or more sequences of one or more instructions contained in the dedicated RAM of these special-purpose processing units. Such instructions can be read into RAM 806 from another computer-readable medium or computer-readable storage medium, such as storage device 810. Execution of the sequences of instructions contained in RAM 806 can cause processor 804 to perform the processes described herein. Alternatively, hard-wired circuitry can be used in place of or in combination with software instructions to implement the present teachings. Thus, implementations of the present teachings are not limited to any specific combination of hardware circuitry and software.

The term “computer-readable medium” (e.g., data store, data storage, storage device, data storage device, etc.) or “computer-readable storage medium” as used herein refers to any media that participates in providing instructions to processor 804 for execution. Such a medium can take many forms, including but not limited to, non-volatile media, volatile media, and transmission media. Examples of non-volatile media can include, but are not limited to, optical, solid state, magnetic disks, such as storage device 810. Examples of volatile media can include, but are not limited to, dynamic memory, such as RAM 806. Examples of transmission media can include, but are not limited to, coaxial cables, copper wire, and fiber optics, including the wires that comprise bus 802.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, any other memory chip or cartridge, or any other tangible medium from which a computer can read.

In addition to computer readable medium, instructions or data can be provided as signals on transmission media included in a communications apparatus or system to provide sequences of one or more instructions to processor 804 of computer system 800 for execution. For example, a communication apparatus may include a transceiver having signals indicative of instructions and data. The instructions and data are configured to cause one or more processors to implement the functions outlined in the disclosure herein. Representative examples of data communications transmission connections can include, but are not limited to, telephone modem connections, wide area networks (WAN), local area networks (LAN), infrared data connections, NFC connections, optical communications connections, etc.

It should be appreciated that the methodologies described herein, flow charts, diagrams, and accompanying disclosure can be implemented using computer system 800 as a standalone device or on a distributed network of shared computer processing resources such as a cloud computing network.

The methodologies described herein may be implemented by various means depending upon the application. For example, these methodologies may be implemented in hardware, firmware, software, or any combination thereof. For a hardware implementation, the processing unit may be implemented within one or more application specific integrated circuits (ASICs), digital signal processors (DSPs), digital signal processing devices (DSPDs), programmable logic devices (PLDs), field programmable gate arrays (FPGAs), processors, graphical processing units (GPUs), tensor processing units (TPUs), controllers, micro-controllers, microprocessors, electronic devices, other electronic units designed to perform the functions described herein, or a combination thereof.

In various embodiments, the methods of the present teachings may be implemented as firmware and/or a software program and applications written in conventional programming languages such as C, C++, Python, etc. If implemented as firmware and/or software, the embodiments described herein can be implemented on a non-transitory computer-readable medium in which a program is stored for causing a computer to perform the methods described above. It should be understood that the various engines described herein can be provided on a computer system, such as computer system 800, whereby processor 804 would execute the analyses and determinations provided by these engines, subject to instructions provided by any one of, or a combination of, the memory components RAM 806, ROM, 808, or storage device 810 and user input provided via input device 814.

### EXAMPLES

### Example 1: Automated Screening of Moderately Severe and Severe Non-Proliferative Diabetic Retinopathy from 7-Field Color Fundus Photographs using Deep Learning

The performance of a deep learning (DL) model employing 7-fields color fundus photographs (7F-CFP) in the automated identification of eyes with moderately severe and severe non-proliferative diabetic retinopathy (NPDR) was assessed among patients with diabetes in a U.S. primary care setting.

Eyes of 55,324 patients with diabetes were analyzed using data collected between 1999 and 2016 (Source: Inoveon Corporation, Oklahoma City, OK). Of these, 13,247 patients were excluded due to a lack of image data, 26 patients were excluded as acquisition test cases, and 4,693 patients were excluded due to the presence of ungradeable images. This left a total of 37,358 patients for assessment using the systems and methods disclosed herein. Diabetic retinopathy (DR) severity and the presence of clinically significant macular edema (CSME) were assessed from 7F-CFP by professional graders at a centralized reading center. DR severity was graded using the Early Treatment Diabetic Retinopathy Study (ETDRS) DR Severity Scale (DRSS).

The dataset was split into 80% for model training, 10% for tuning and 10% for testing, for a total of 29,890 patients, 3,732 patients and 3,736 patients, respectively. Table 1 shows demographic information for patients included in the dataset. Table 2 shows clinical information for patients included in the dataset. Table 3 shows DRSS scores for patients included in the dataset.

TABLE 1
Patient demographic information

| Demographic Characteristics | Analysis Population (N = 37,358) | |
|---|---|---|
| Median age, years (range) | 61 | (6-85) |
| Sex, n (%) | | |
| Male | 18,922 | (75.8) |
| Female | 6,042 | (24.2) |
| Unknown | 12,394 | |
| Race, n % | | |
| White | 3,271 | (54.25) |
| Native American | 1,925 | (31.94) |
| African American/Black | 745 | (12.35) |
| Asian | 65 | (1.08) |
| Other | 23 | (0.38) |

| | |
|---|---|
| Unknown | 31,328 |

TABLE 2
Patient clinical information

| Clinical Characteristics | Analysis Population (N = 37,358) | |
|---|---|---|
| Diabetes type, n (%) | | |
| Type 1 | 1,245 | (7.6) |
| Type 2 | 15,116 | (92.28) |
| Gestational | 20 | (0.12) |

| | |
|---|---|
| Unknown | 20,977 |

TABLE 3
Patient DRSS scores

| DRSS | Analysis Population (N = 37,358) | |
|---|---|---|
| 10 or 12 | 22,339 | (59.79%) |
| 14, 15, or 20 | 7,780 | (20.83%) |
| 35 | 4,875 | (13.05%) |
| 43 | 1,112 | (2.98%) |
| 47 | 530 | (1.42%) |
| 53 | 297 | (0.80%) |
| 60 or 61 | 235 | (0.63%) |
| 65 | 134 | (0.36%) |
| 71 | 48 | (0.13%) |
| 75 | 6 | (<0.1%) |
| 81 | 2 | (<0.1%) |

A deep learning Inception V3 model with transfer-learning was trained at the image level on all 7 fields of view (including stereoscopy) to classify patients as either having or not having a DRSS in the range from 47 to 53. Predictions were averaged over all fields of view to provide a prediction at the eye level for each patient. Model performance was determined based on area under the receiver operating characteristic curve (AUROC), specificity, sensitivity and positive predictive value.

A model was selected based on performance on the tuning set, as well as a desired cutoff for specificity and sensitivity to maximize the Youden-index. The model performed well on the testing set to identify patients with DRSS in the range of 47 to 53 at an AUROC of 0.988 (95% CI, 0.9872-0.9879), precision of 0.57 (95% CI, 0.56-0.58), sensitivity of 0.9639 (95% CI, 0.9628-0.9655), specificity of 0.9624 (95% CI, 0.9621-0.9625), positive predictive value of 0.368 (95% CI, 0.366-0.370), and negative predictive value of 0.999 (95% CI, 0.9991-0.0002). Additionally, for members of the testing set who had more than mild DR, the model achieved an AUROC of 0.93 (95% CI, 0.93-0.94), precision of 0.574 (95% CI, 0.567-0.58), sensitivity of 0.9639 (95% CI, 0.9624-0.9652), specificity of 0.7912 (95% CI 0.7901-0.7923), positive predictive value of 0.376 (95% CI, 0.3743-0.3786), and negative predictive value of 0.994 (95% CI, 0.9938-0.9943).

These results show that machine learning (and in particular DL) can support automated identification of eyes with DRSS in the range of 47 to 53. Such a model can support the screening through the identification of patients at risk of progression for preventive clinical trials. Additionally, it can aid with patient screening in clinical practice.

### Conclusion

While the present teachings are described in conjunction with various embodiments, it is not intended that the present teachings be limited to such embodiments. On the contrary, the present teachings encompass various alternatives, modifications, and equivalents, as will be appreciated by those of skill in the art.

For example, the flowcharts and block diagrams described above illustrate the architecture, functionality, and/or operation of possible implementations of various method and system embodiments. Each block in the flowcharts or block diagrams may represent a module, a segment, a function, a portion of an operation or step, or a combination thereof. In some alternative implementations of an embodiment, the function or functions noted in the blocks may occur out of the order noted in the figures. For example, in some cases, two blocks shown in succession may be executed substantially concurrently or integrated in some manner. In other cases, the blocks may be performed in the reverse order. Further, in some cases, one or more blocks may be added to replace or supplement one or more other blocks in a flowchart or block diagram.

Thus, in describing the various embodiments, the specification may have presented a method and/or process as a particular sequence of steps. However, to the extent that the method or process does not rely on the particular order of steps set forth herein, the method or process should not be limited to the particular sequence of steps described, and one skilled in the art can readily appreciate that the sequences may be varied and still remain within the spirit and scope of the various embodiments.

### RECITATION OF EMBODIMENTS

Embodiment 1. A method for evaluating diabetic retinopathy (DR) severity, the method comprising:
receiving input data comprising at least color fundus imaging data for an eye of a subject; performing at least one image standardization procedure on the color fundus imaging data;
generating a set of standardized image data; and
generating, using at least the standardized image data, a metric indicating a probability that a score for DR severity in the eye of the subject falls within a selected range.

Embodiment 2. The method of Embodiment 1, wherein the color fundus imaging data comprises a plurality of fields of view, each field of view comprising a color fundus image.

Embodiment 3. The method of Embodiment 1 or 2, further comprising:
determining a gradeability of the color fundus imaging data based on a number of fields of view associated with the color fundus imaging data, the gradeability indicating an indication that the color fundus imaging data does or does not contain a predetermined number of fields of view.

Embodiment 4. The method of any one of Embodiments 1-3, wherein the selected range denotes a mild to moderate DR, a mild to moderately severe DR, a mild to severe DR, a moderate to moderately severe DR, a moderately severe to severe DR, a moderate to severe DR, a more than moderate DR, a more than mild DR, a more than moderately severe DR, or a more than severe DR.

Embodiment 5. The method of any one of Embodiments 1-4, wherein the selected range comprises a portion of a Diabetic Retinopathy Severity Scale (DRSS) between and including 35 and 43, between and including 35 and 47, between and including 35 and 53, between and including 43 and 47, between and including 47 and 53, between and including 43 and 53, at least 35, at least 43, at least 47, or at least 53.

Embodiment 6. The method of any one of Embodiments 1-5, further comprising:
predicting the score for the DR severity in the eye using the received color fundus imaging data.

Embodiment 7. The method any one of Embodiments 1-6, wherein the metric comprises a predicted DR severity score for the eye.

Embodiment 8. The method of any one of Embodiments 1-7, wherein the at least one image standardization procedure comprises one or more of: a field detection procedure, a central cropping procedure, a foreground extraction procedure, a region extraction procedure, a central region extraction procedure, an adaptive histogram equalization (AHE) procedure, and a contrast limited AHE (CLAHE) procedure.

Embodiment 9. The method of any one of Embodiments 1-8, wherein the input data further comprises one or more of: baseline demographic characteristics associated with the subject and baseline clinical characteristics associated with the subject; and wherein the generating the metric further comprises generating the metric using one or more of the baseline demographic characteristics and the baseline clinical characteristics.

Embodiment 10. The method of any one of Embodiments 1-9, wherein the generating the metric comprises generating the metric using a neural network system.

Embodiment 11. The method of Embodiment 10, further comprising:
training the neural network system using a training dataset comprising at least graded color fundus imaging data associated with a plurality of training subjects.

Embodiment 12. The method of Embodiment 11, wherein the training the neural network system further comprises training the neural network using one or more of: baseline demographic characteristics associated with the plurality of training subjects and baseline clinical characteristics associated with the plurality of training subjects.

Embodiment 13. A system for evaluating diabetic retinopathy (DR) severity, the system comprising:
a non-transitory memory; and
one or more processors coupled to the non-transitory memory and configured to read instructions from the non-transitory memory to cause the system to perform operations comprising:
receiving input data comprising at least color fundus imaging data for an eye of a subject;
performing at least one image standardization procedure on the color fundus imaging data;
generating a set of standardized image data; and
generating, using at least the standardized image data, a metric indicating a probability that a score for DR severity in the eye of the subject falls within a selected range.

Embodiment 14. The system of Embodiment 13, wherein the color fundus imaging data comprises a plurality of fields of view, each field of view comprising a color fundus image.

Embodiment 15. The system of Embodiment 13 or 14, wherein the operations further comprise:
determining a gradeability of the color fundus imaging data based on a number of fields of view associated with the color fundus imaging data, the gradeability indicating an indication that the color fundus imaging data does or does not contain a predetermined number of fields of view.

Embodiment 16. The system of any one of Embodiments 13-15, wherein the selected range denotes a mild to moderate DR, a mild to moderately severe DR, a mild to severe DR, a moderate to moderately severe DR, a moderately severe to severe DR, a moderate to severe DR, a more than mild DR, a more than moderate DR, a more than moderately severe DR, or a more than severe DR.

Embodiment 17. The system of any one of Embodiments 13-16, wherein the selected range comprises a portion of a Diabetic Retinopathy Severity Scale (DRSS) between and including 35 and 43. between and including 35 and 47, between and including 35 and 53, between and including 43 and 47, between and including 47 and 53, between and including 43 and 53, at least 35, at least 43, at least 47, or at least 53.

Embodiment 18. The system of any one of Embodiments 13-17, wherein the operations further comprise:
predicting the score for the DR severity in the eye using the received color fundus imaging data.

Embodiment 19. The system of any one of Embodiments 13-18, wherein the metric comprises a predicted DR severity score for the eye.

Embodiment 20. The system of any one of Embodiments 13-19, wherein the at least one image standardization procedure comprises one or more of: a field detection procedure, a central cropping procedure, a foreground extraction procedure, a region extraction procedure, a central region extraction procedure, an adaptive histogram equalization (AHE) procedure, and a contrast limited AHE (CLAHE) procedure.

Embodiment 21. The system of any one of Embodiments 13-20, wherein the input data further comprises one or more of: baseline demographic characteristics associated with the subject and baseline clinical characteristics associated with the subject; and wherein the generating the metric further comprises generating the metric using one or more of the baseline demographic characteristics and the baseline clinical characteristics.

Embodiment 22. The system of any one of Embodiments 13-21, wherein the generating the metric comprises generating the metric using a neural network system.

Embodiment 23. The system of Embodiment 22, wherein the operations further comprise:
training the neural network system using a training dataset comprising at least graded color fundus imaging data associated with a plurality of training subjects.

Embodiment 24. The system of Embodiment 23, wherein the training the neural network system further comprises training the neural network using one or more of: baseline demographic characteristics associated with the plurality of training subjects and baseline clinical characteristics associated with the plurality of training subjects.

Embodiment 25. A non-transitory, machine-readable medium having stored thereon machine-readable instructions executable to cause a system to perform operations comprising:
receiving input data comprising at least color fundus imaging data for an eye of a subject;
performing at least one image standardization procedure on the color fundus imaging data;
generating a set of standardized image data; and
generating, using at least the standardized image data, a metric indicating a probability that a score for DR severity in the eye of the subject falls within a selected range.

Embodiment 26. The non-transitory, machine-readable medium of Embodiment 25, wherein the color fundus imaging data comprises a plurality of fields of view, each field of view comprising a color fundus image.

Embodiment 27. The non-transitory, machine-readable medium of Embodiment 25 or 26, wherein the operations further comprise:
determining a gradeability of the color fundus imaging data based on a number of fields of view associated with the color fundus imaging data, the gradeability indicating an indication that the color fundus imaging data does or does not contain a predetermined number of fields of view.

Embodiment 28. The non-transitory, machine-readable medium of any one of Embodiments 25-27, wherein the selected range denotes a mild to moderate DR, a mild to moderately severe DR, a mild to severe DR, a moderate to moderately severe DR, a moderately severe to severe DR, a moderate to severe DR, a more than mild DR, a more than moderate DR, a more than moderately severe DR, or a more than severe DR.

Embodiment 29. The non-transitory, machine-readable medium of any one of Embodiments 25-28, wherein the selected range comprises a portion of a Diabetic Retinopathy Severity Scale (DRSS) between and including 35 and 43, between and including 35 and 47, between and including 35 and 53, between and including 43 and 47, between and including 47 and 53, between and including 43 and 53, at least 35, at least 43, at least 47, or at least 53.

Embodiment 30. The non-transitory, machine-readable medium of any one of Embodiments 25-29, wherein the operations further comprise:
predicting the score for the DR severity in the eye using the received color fundus imaging data.

Embodiment 31. The non-transitory, machine-readable medium of any one of Embodiments 25-30, wherein the metric comprises a predicted DR severity score for the eye.

Embodiment 32. The non-transitory, machine-readable medium of any one of Embodiments 25-31, wherein the at least one image standardization procedure comprises one or more of: a field detection procedure, a central cropping procedure, a foreground extraction procedure, a region extraction procedure, a central region extraction procedure, an adaptive histogram equalization (AHE) procedure, and a contrast limited AHE (CLAHE) procedure.

Embodiment 33. The non-transitory, machine-readable medium of any one of Embodiments 25-32, wherein the input data further comprises one or more of: baseline demographic characteristics associated with the subject and baseline clinical characteristics associated with the subject; and wherein the generating the metric further comprises generating the metric using one or more of the baseline demographic characteristics and the baseline clinical characteristics.

Embodiment 34. The method of any one of Embodiments 25-33, wherein the generating the metric comprises generating the metric using a neural network system.

Embodiment 35. The non-transitory, machine-readable medium of Embodiment 34, wherein the operations further comprise:
training the neural network system using a training dataset comprising at least graded color fundus imaging data associated with a plurality of training subjects.

Embodiment 36. The non-transitory, machine-readable medium of Embodiment 35, wherein the training the neural network system further comprises training the neural network using one or more of: baseline demographic characteristics associated with the plurality of training subjects and baseline clinical characteristics associated with the plurality of training subjects.

Embodiment 37. A method for evaluating diabetic retinopathy (DR) severity, the method comprising:
determining one or more DR severity scores, each score associated with a DR severity level;
determining a plurality of DR severity classifications, each classification denoted by a range or a set of DR severity threshold scores;
receiving input data comprising at least color fundus imaging data for an eye of a subject;
determining, from the received input data, a metric indicating a probability that a score for DR severity in the eye of the subject falls within a selected range; and
classifying the eye of the received input data into a DR severity classification of the plurality of DR severity classifications based on the metric.

Embodiment 38. The method of Embodiment 37, further comprising: determining the range or set of DR threshold scores, each DR threshold score indicating a minimum or maximum score corresponding to a DR severity classification of the plurality of DR severity classifications.

Embodiment 39. The method of Embodiment 37 or 38, wherein the at least one DR severity classification denotes a moderate to moderately severe DR, a moderately severe to severe DR, or a moderate to severe DR.

Embodiment 40. The method of any one of Embodiments 37-39, wherein the at least one range or set of DR severity threshold scores comprises a portion of a Diabetic Retinopathy Severity Scale (DRSS) between and including 43 and 47, between and including 47 and 53, or between and including 43 and 53.

Embodiment 41. The method of any one of Embodiments 37-40, further comprising:
predicting the score for the DR severity in the eye using the received color fundus imaging data.

Embodiment 42. The method of any one of Embodiments 37-41, wherein the metric comprises a predicted DR severity score for the eye.

Embodiment 43. The method of any one of Embodiments 37-42, wherein the input data further comprises one or more of: baseline demographic characteristics associated with the subject and baseline clinical characteristics associated with the subject; and wherein the generating the metric further comprises generating the metric using one or more of the baseline demographic characteristics and the baseline clinical characteristics.

Embodiment 44. The method of any one of Embodiments 37-43, wherein the generating the metric comprises generating the metric using a neural network system.

Embodiment 45. The method of Embodiment 44, further comprising:
training the neural network system using a training dataset comprising at least graded color fundus imaging data associated with a plurality of training subjects.

Embodiment 46. The method of Embodiment 45, wherein the training the neural network system further comprises training the neural network using one or more of: baseline demographic characteristics associated with the plurality of training subjects and baseline clinical characteristics associated with the plurality of training subjects.

Embodiment 47. A system for evaluating diabetic retinopathy (DR) severity, the system comprising:
a non-transitory memory; and
one or more processors coupled to the non-transitory memory and configured to read instructions from the non-transitory memory to cause the system to perform operations comprising:
   receiving a determination of one or more DR severity scores, each score associated with a DR severity level;
   receiving a determination of a plurality of DR severity classifications, each classification denoted by a range or a set of DR severity threshold scores;
   receiving input data comprising at least color fundus imaging data for an eye of a subject;
   determining, from the received input data, a metric indicating a probability that a score for DR severity in the eye of the subject falls within a selected range; and
   classifying the eye of the received input data into a DR severity classification of the plurality of DR severity classifications based on the metric.

Embodiment 48. The system of Embodiment 47, wherein the operations further comprise: receiving a determination of the range or set of DR threshold scores, each DR threshold score indicating a minimum or maximum score corresponding to a DR severity classification of the plurality of DR severity classifications.

Embodiment 49. The system of Embodiment 47 or 48, wherein the at least one DR severity classification denotes a moderate to moderately severe DR, a moderately severe to severe DR, or a moderate to severe DR.

Embodiment 50. The system of any one of Embodiments 47-49, wherein the at least one range or set of DR severity threshold scores comprises a portion of a Diabetic Retinopathy Severity Scale (DRSS) between and including 43 and 47, between and including 47 and 53, or between and including 43 and 53.

Embodiment 51. The system of any one of Embodiments 47-50, wherein the operations further comprise:
predicting the score for the DR severity in the eye using the received color fundus imaging data.

Embodiment 52. The system of any one of Embodiments 47-51, wherein the metric comprises a predicted DR severity score for the eye.

Embodiment 53. The system of any one of Embodiments 47-52, wherein the input data further comprises one or more of: baseline demographic characteristics associated with the subject and baseline clinical characteristics associated with the subject; and wherein the generating the metric further comprises generating the metric using one or more of the baseline demographic characteristics and the baseline clinical characteristics.

Embodiment 54. The system of any one of Embodiments 47-53, wherein the generating the metric comprises generating the metric using a neural network system.

Embodiment 55. The system of Embodiment 54, wherein the operations further comprise:
training the neural network system using a training dataset comprising at least graded color fundus imaging data associated with a plurality of training subjects.

Embodiment 56. The system of Embodiment 55, wherein the training the neural network system further comprises training the neural network using one or more of: baseline demographic characteristics associated with the plurality of training subjects and baseline clinical characteristics associated with the plurality of training subjects.

Embodiment 57. A non-transitory, machine-readable medium having stored thereon machine-readable instructions executable to cause a system to perform operations comprising:
receiving a determination of one or more DR severity scores, each score associated with a DR severity level;
receiving a determination of a plurality of DR severity classifications, each classification denoted by a range or a set of DR severity threshold scores;
receiving input data comprising at least color fundus imaging data for an eye of a subject;
determining, from the received input data, a metric indicating a probability that a score for DR severity in the eye of the subject falls within a selected range; and
classifying the eye of the received input data into a DR severity classification of the plurality of DR severity classifications based on the metric.

Embodiment 58. The non-transitory, machine-readable medium of Embodiment 57, wherein the operations further comprise: receiving a determination of the range or set of DR threshold scores, each DR threshold score indicating a minimum or maximum score corresponding to a DR severity classification of the plurality of DR severity classifications.

Embodiment 59. The non-transitory, machine-readable medium of Embodiment 57 or 58, wherein the at least one DR severity classification denotes a moderate to moderately severe DR, a moderately severe to severe DR, or a moderate to severe DR.

Embodiment 60. The non-transitory, machine-readable medium of any one of Embodiments 57-59, wherein the at least one range or set of DR severity threshold scores comprises a portion of a Diabetic Retinopathy Severity Scale (DRSS) between and including 43 and 47, between and including 47 and 53, or between and including 43 and 53.

Embodiment 61. The non-transitory, machine-readable medium of any one of Embodiments 57-60, wherein the operations further comprise:
predicting the score for the DR severity in the eye using the received color fundus imaging data.

Embodiment 62. The non-transitory, machine-readable medium of any one of Embodiments 57-61, wherein the metric comprises a predicted DR severity score for the eye.

Embodiment 63. The non-transitory, machine-readable medium of any one of Embodiments 57-62, wherein the input data further comprises one or more of: baseline demographic characteristics associated with the subject and baseline clinical characteristics associated with the subject; and wherein the generating the metric further comprises generating the metric using one or more of the baseline demographic characteristics and the baseline clinical characteristics.

Embodiment 64. The non-transitory, machine-readable medium of any one of Embodiments 57-63, wherein the generating the metric comprises generating the metric using a neural network system.

Embodiment 65. The non-transitory, machine-readable medium of Embodiment 64, wherein the operations further comprise:
training the neural network system using a training dataset comprising at least graded color fundus imaging data associated with a plurality of training subjects.

Embodiment 66. The non-transitory, machine-readable medium of Embodiment 65, wherein the training the neural network system further comprises training the neural network using one or more of: baseline demographic characteristics associated with the plurality of training subjects and baseline clinical characteristics associated with the plurality of training subjects.

1. A method for evaluating diabetic retinopathy (DR) severity, the method comprising:
determining one or more DR severity scores, each score associated with a DR severity level;
determining a plurality of DR severity classifications, each classification denoted by a range or a set of DR severity threshold scores;
receiving input data comprising at least color fundus imaging data for an eye of a subject;
determining, from the received input data, a metric indicating a probability that a score for DR severity in the eye of the subject falls within a selected range; and
classifying the eye of the received input data into a DR severity classification of the plurality of DR severity classifications based on the metric.

2. The method of claim 1, further comprising: determining the range or set of DR threshold scores, each DR threshold score indicating a minimum or maximum score corresponding to a DR severity classification of the plurality of DR severity classifications.

3. The method of claim 1, wherein the at least one DR severity classification denotes a moderate to moderately severe DR, a moderately severe to severe DR, or a moderate to severe DR.

4. The method of claim 1, wherein the at least one range or set of DR severity threshold scores comprises a portion of a Diabetic Retinopathy Severity Scale (DRSS) between and including 43 and 47, between and including 47 and 53, or between and including 43 and 53.

5. The method of claim 1, wherein the input data further comprises one or more of: baseline demographic characteristics associated with the subject and baseline clinical characteristics associated with the subject; and wherein the generating the output further comprises generating the output using one or more of the baseline demographic characteristics and the baseline clinical characteristics.

6. The method of claim 1, wherein the generating the metric comprises generating the metric using a neural network system.

7. The method of claim 6, further comprising:
training the neural network system using a training dataset comprising at least graded color fundus imaging data associated with a plurality of training subjects.

8. The method of claim 7, wherein the training the neural network system further comprises training the neural network using one or more of: baseline demographic characteristics associated with the plurality of training subjects and baseline clinical characteristics associated with the plurality of training subjects.

9. A system for evaluating diabetic retinopathy (DR) severity, the system comprising:
a non-transitory memory; and
one or more processors coupled to the non-transitory memory and configured to read instructions from the non-transitory memory to cause the system to perform operations comprising:
receiving a determination of one or more DR severity scores, each score associated with a DR severity level;
receiving a determination of a plurality of DR severity classifications, each classification denoted by a range or a set of DR severity threshold scores;
receiving input data comprising at least color fundus imaging data for an eye of a subject;
determining, from the received input data, a metric indicating a probability that a score for DR severity in the eye of the subject falls within a selected range; and
classifying the eye of the received input data into a DR severity classification of the plurality of DR severity classifications based on the metric.

10. The system of claim 9, wherein the operations further comprise: receiving a determination of the range or set of DR threshold scores, each DR threshold score indicating a minimum or maximum score corresponding to a DR severity classification of the plurality of DR severity classifications.

11. The system of claim 9, wherein the at least one DR severity classification denotes a moderate to moderately severe DR, a moderately severe to severe DR, or a moderate to severe DR.

12. The system of claim 9, wherein the at least one range or set of DR severity threshold scores comprises a portion of a Diabetic Retinopathy Severity Scale (DRSS) between and including 43 and 47, between and including 47 and 53, or between and including 43 and 53.

13. The system of claim 9, wherein the input data further comprises one or more of: baseline demographic characteristics associated with the subject and baseline clinical characteristics associated with the subject; and wherein the generating the output further comprises generating the output using one or more of the baseline demographic characteristics and the baseline clinical characteristics.

14. The system of claim 9, wherein the generating the metric comprises generating the metric using a neural network system.

15. A non-transitory, machine-readable medium having stored thereon machine-readable instructions executable to cause a system to perform operations comprising:
receiving a determination of one or more DR severity scores, each score associated with a DR severity level;
receiving a determination of a plurality of DR severity classifications, each classification denoted by a range or a set of DR severity threshold scores;
receiving input data comprising at least color fundus imaging data for an eye of a subject;
determining, from the received input data, a metric indicating a probability that a score for DR severity in the eye of the subject falls within a selected range; and
classifying the eye of the received input data into a DR severity classification of the plurality of DR severity classifications based on the metric.

16. The non-transitory, machine-readable medium of claim 15, wherein the operations further comprise: receiving a determination of the range or set of DR threshold scores, each DR threshold score indicating a minimum or maximum score corresponding to a DR severity classification of the plurality of DR severity classifications.

17. The non-transitory, machine-readable medium of claim 15, wherein the at least one DR severity classification denotes a moderate to moderately severe DR, a moderately severe to severe DR, or a moderate to severe DR.

18. The non-transitory, machine-readable medium of claim 15, wherein the at least one range or set of DR severity threshold scores comprises a portion of a Diabetic Retinopathy Severity Scale (DRSS) between and including 43 and 47, between and including 47 and 53, or between and including 43 and 53.

19. The non-transitory, machine-readable medium of claim 15, wherein the input data further comprises one or more of: baseline demographic characteristics associated with the subject and baseline clinical characteristics associated with the subject; and wherein the generating the output further comprises generating the output using one or more of the baseline demographic characteristics and the baseline clinical characteristics.

20. The non-transitory, machine-readable medium of claim 15, wherein the generating the metric comprises generating the metric using a neural network system.
